SEQUENCE LISTING (13 sequences)

SEQ ID NO: 1 - protein, 33 aa, Artificial Sequence
  Wild type peptide sequence representing the human-derived S1P4 extracellular Loop 1
TGAAYLANVLLSGARTFRLAPAQWFLREGLLFT

SEQ ID NO: 2 - protein, 33 aa, Artificial Sequence
  Loop 1 B peptide sequence representing the human derived S1P4 extracellular Loop 1
TGACYLANVLLSGARTFRLAPAQWFLREGCLFT

SEQ ID NO: 3 - protein, 38 aa, Artificial Sequence
  Wild type peptide sequence representing the human derived S1P4 extracellular Loop 2
AALLGMLPLLGWNCLCAFDRCSSLLPLYSKRYILFCLV

SEQ ID NO: 4 - protein, 38 aa, Artificial Sequence
  Model peptide 2 A sequence representing the human derived S1P4 extracellular Loop 2
AALCGMLPLLGWNALAAFDRASSLLPLYSKRYILFCLV

SEQ ID NO: 5 - protein, 39 aa, Artificial Sequence
  Wild type peptide sequence representing the human derived S1P4 extracellular Loop 3
FLVCWGPLFGLLLADVFGSNLWAQEYLRGMDWILALAVL

SEQ ID NO: 6 - protein, 39 aa, Artificial Sequence
  Model peptide 3 sequence representing the human derived S1P4 extracellular Loop 3
FLVCWGPLFGLLLADVFGSNLWAQEYLRGMDWILACAVL

SEQ ID NO: 7 - protein, 33 aa, Artificial Sequence; identical to SEQ ID NO: 1
  Modeled wild type peptide sequence of human derived S1P4 extracellular Loop 1

SEQ ID NO: 8 - protein, 33 aa, Artificial Sequence; identical to SEQ ID NO: 2
  Modeled Loop 1 B peptide sequence of human derived S1P4 extracellular Loop 1

SEQ ID NO: 9 - protein, 29 aa, Artificial Sequence
  Modeled cyclic peptide for use as human derived LPA 2 cyclic receptor mimic for extracellular Loop 1
AYLFLMFHTGPRTARLSLEGWFLRQGLLD

SEQ ID NO: 10 - protein, 35 aa, Artificial Sequence
  Modeled cyclic peptide for use as human derived LPA 2 cyclic receptor mimic for extracellular Loop 2
LGLLPAHSWHCLCALDRCSRMAPLLSRSYLAVWAL

SEQ ID NO: 11 - protein, 32 aa, Artificial Sequence
  Modeled cyclic peptide for use as human derived LPA 2 cyclic receptor mimic for extracellular Loop 3
CWTPGQVVLLLDGLGCESCNVLAVEKYFLLLA

SEQ ID NO: 12 - protein, 29 aa, Artificial Sequence
  Intramolecular disulfide constraint for modeled cyclic peptide of human derived LPA 2 extracellular Loop 1
AYLFLMFCTGPRTARLSLCGWFLRQGLLD

SEQ ID NO: 13 - protein, 35 aa, Artificial Sequence
  Intramolecular disulfide constraint for modeled cyclic peptide of human derived LPA 2 extracellular Loop 2
LGLLPAHCWHSLSALDRSSRMAPLLCRSYLAVWAL

(19) United States
(12) Patent Application Publication
(10) Pub. No.: US 2005/0079549 A1
(43) Pub. Date: Apr. 14, 2005

(54) Methods for modeling GPCRs and for producing ligand blocking and receptor activating antibodies for same

(76) Inventor: John Castracane, Groton, MA (US)

Correspondence Address: MIRICK O'CONNELL;MIRICK O'CONNELL, DEMALLIE & LOUGEE, LLP, 100 FRONT STREET, WORCESTER, MA, 01608-1477, US

(21) Appl. No.: 10/897,170

(22) Filed: Jul. 22, 2004

Related U.S. Application Data
(60) Provisional application No. 60/489,547, filed on Jul. 23, 2003.

(51) Int. Cl. G01N 33/53; G06F 19/00; G01N 33/48; G01N 33/50

(52) U.S. Cl. 435/7.1; 702/19

(57) ABSTRACT

A method for modeling G protein coupled receptors (GPCR) and producing conformationally constrained peptides or fragments thereof that generally includes the steps of: providing a molecular model of a GPCR and identifying a peptide sequence therein, having at least one peptide residue involved in ligand binding; identifying a plurality of amino acid sequences extracellular and proximal to at least one transmembrane domain and at least one extracellular loop; identifying at least one amino acid on said loop as an optimal location for a conformational constraint in said extracellular loop; mutating said identified amino acid to cysteine if not already cysteine; covalently connecting one or more linkers to said cysteine to conformationally constrain said peptide; and characterizing said constrained peptide using nuclear magnetic resonance (NMR) to verify a stable tertiary structure having conformations substantially similar to overlapping regions of a molecular model of said modeled GPCR containing said peptide.

### CROSS-REFERENCE

This is a continuation-in-part of U.S. Provisional Application Ser. No. 60/489,547 filed on Jul. 23, 2003.

### FIELD OF THE INVENTION

The invention relates to the field of drug discovery and the identification and production of ligand blocking and receptor activating antibodies with potential therapeutic application.

### BACKGROUND OF THE INVENTION

The GPCR gene family is the largest known receptor family. GPCRs are a superfamily of membrane-spanning proteins having seven alpha-helical transmembrane domains that are involved in the transduction of chemical signals across cellular membranes. Approximately 1-2% of human genes are thought to code for GPCRs, and up to 60% of the modern pharmacopoeia targets them. (Hibert, M. F., Trumpp-Kallmeyer, S., Bruinvels, A. & Hoflack, J. Three-Dimensional Models of Neurotransmitter G-Binding Protein-Coupled Receptors. Mol. Pharmacol. 40, 8-15 (1991)). The G-protein-coupled receptors (GPCRs) are transducers of extracellular messages and they enable tissues to respond to a wide array of signalling molecules. Most of the endogenous ligands are small and numerous GPCRs are targets of important drugs in use today. Members of the Edg family have been implicated in ovarian cancer, breast cancer, prostate cancer, neuronal development, myocardial development and tumor angiogenesis. GPCR research is a task of prime importance.

Specific GPCR blocking antibodies and activating antibodies are not currently available for the majority of GPCRs. In basic research and drug discovery specific blocking antibodies are needed to identify the receptors mediating various biological responses. In a similar manner, specific activating antibodies would be very useful in the study of the signal transduction pathways of the various GPCRs as the anti-FAS monoclonal CH11 was used to study apoptosis pathways and also to identify the ligand of this death receptor.

Computer modeling in general has enormous potential application to research in the medical sciences, as it has proven very difficult to develop antibody reagents with broad utility for use in GPCR research by conventional means, computer modeling is a fresh approach to antibody development. Antibodies specific for GPCRs have three important commercial applications. First, they are powerful tools that can make possible the development of assays for use in drug discovery efforts, basic research and diagnostics. Such assays will provide evidence for the involvement of receptors in normal or abnormal physiology. Antibodies could find uses in the investigation of GPCRs by flow cytometry, in situ binding studies/immunohistochemistry, western blotting, immunoprecipitation and the development of diagnostic tests. Second, specific antibodies against GPCR have the potential to block ligand binding or otherwise block ligand induced signal transduction and thus receptor activation, thus having commercial therapeutic applications. Third, antibodies that activate GPCR also have therapeutic applications. Thus the validation of a novel approach for the generation of antibodies against GPCRs has enormous commercial potential.

It has not previously proven possible to easily produce blocking antibodies to GPCRs. The use of conventionally identified and produced peptide immunogens has yielded antibodies of restricted utility and their use in multiple assay formats has proven impractical if not impossible. (Goetzl, E. J., Dolezalova, H., Kong, Y. & Zeng, L. Dual Mechanisms for Lysophospholipid Induction of Proliferation of Human Breast Carcinoma Cells. Cancer Res. 59, 4732-4737 (1999)). The development of monoclonal antibodies to conventional peptide immunogens is not a practical solution, as these antibodies generally will only recognize an immunogen once it has been denatured, as in western blotting. The frequency of useful antibodies arising from the use conventional immunogens is very low.

Edg receptors regulate numerous biological effects, including angiogenesis, cell proliferation and cellular motility. (Motohashi, K., Shibata, S., Ozaki, Y., Yatomi, Y. & Igarashi, Y. Identification of Lysophospholipid Receptors in Human Platelets: the Relation of Two Agonists, Lysophosphatidic Acid and Sphingosine 1-Phosphate. FEBS Lett. 468, 189-193 (2000); An, S., Zheng, Y. & Bleu, T. Sphingosine 1-Phosphate-induced Cell Proliferation, Survival, and Related Signaling Events Mediated by G Protein-coupled Receptors Edg3 and Edg5. J. Biol. Chem. 275, 288-296 (2000); Ancellin, N. & Hla, T. Differential Pharmacological Properties and Signal Transduction of the Sphingosine 1-Phosphate Receptors EDG-1, EDG-3, and EDG-5. J. Biol. Chem. 274, 18997-19002 (1999); Goetzl, E. J. & An, S. Diversity of Cellular Receptors and Functions for the Lysophospholipid Growth Factors Lysophosphatidic Acid and Sphingosine-1-phosphate. FASEB J. 12, 1589-1598 (1998); Wang, F. et al. Sphingosine 1-Phosphate Stimulates Cell Migration through a Gᵢ-coupled Cell Surface Receptor, A Potential Involvement in Angiogenesis. J. Biol. Chem. 274, 35343-35350 (1999)).

The availability of specific antibodies for each of the Edg receptors will facilitate research in these fields. Additionally, Edg receptors are true therapeutic targets. Hu et al. report, with Napoleone Ferrara, that they have unraveled a novel pathway for VEGF expression in ovarian cancer. (Journal of the National Cancer Institute, Vol. 93, No. 10, May 16, 2001). They show that lysophosphatidic acid (LPA) in ovarian cancer ascites fluid binds the LPA2 receptor, which is expressed in ovarian cancer cells but not in normal ovarian epithelial cells. The result of ligand binding is increased VEGF expression by the cancer cells. Activation of the LPA2 receptor increased expression of the VEGF promoter by a mechanism that is mediated through c-Jun and c-Fos and differs qualitatively from hypoxia-mediated VEGF expression through increases in the half-life of VEGF messenger RNA.

These results suggest that LPA2 may provide a new target for therapy in ovarian cancer. This report suggests the possible development of a novel angiogenesis inhibitor that could shut off the angiogenic switch in ovarian cancer or at least control one aspect of this switch. Antiangiogenic therapy could also reduce ascites, as has been demonstrated previously in animals. Because LPA is mitogenic for ovarian cancer cells, an LPA2 inhibitor could also directly block cancer cell proliferation. Impact of oncogenes in tumor angiogenesis: Mutant K-ras up-regulation of vascular endothelial growth factor vascular permeability factor is necessary, but not sufficient for tumorigenicity of human colorectal carcinoma cells. (Proc. Natl. Acad. Sci. USA Vol. 95, pp. 3609-3614, March 1998; Yu-Long Hu, Meng-Kian Tee, Edward J. Goetzl, Nelly Auersperg, Gordon B. Mills, Napoleone Ferrara, Robert B. Jaffe; Lysophosphatidic Acid Induction of Vascular Endothelial Growth Factor Expression in Human Ovarian Cancer Cells. Journal of the National Cancer Institute, Vol. 93, No. 10, May 16, 2001).

Various members of the Edg family of GPCRs have been demonstrated to have potential involvement in numerous clinical conditions. Edg receptors are potential therapeutic targets in connection with angiogensis, (Judah Folkman, Journal of the National Cancer Institute, Vol. 93, No. 10, 734-735, May 16, 2001 (2001 Oxford University Press), A New Link in Ovarian Cancer Angiogenesis: Lysophosphatidic Acid and Vascular Endothelial Growth Factor Expression), nervous system (Beer, M. S. et al. in Lysophospholipids and Eicosanoids in Biology and Pathophysiology (eds. Goetzl, E. J. & Lynch, K. R.) 118-131 (New York Academy of Sciences, New York, 2000)), prostate cancer, (Im, D.-S. et al. Molecular Cloning and Characterization of a Lysophosphatidic Acid Receptor, Edg-7, Expressed in Prostate. Mol. Pharmacol. 57, 753-759 (2000)), breast cancer, (Goetzl, E. J., Dolezalova, H., Kong, Y. & Zeng, L. Dual Mechanisms for Lysophospholipid Induction of Proliferation of Human Breast Carcinoma Cells. Cancer Res. 59, 4732-4737 (1999)), ovarian cancer, (Furui, T. et al. Overexpression of edg-2/vzg-1 Induces Apoptosis and Anoikis in Ovarian Cancer Cells in a Lysophosphatidic Acid-Independent Manner. Clin. Cancer Res. 5, 4308-4318 (1999); Goetzl, E. J. et al. Distinctive Expression and Functions of the Type 4 Endothelial Differentiation Gene-encoded G Protein-coupled Receptor for Lysophosphatidic Acid in Ovarian Cancer. Cancer Res. 59, 5370-5375 (1999); Fang, X. et al. in Lysophospholipids and Eicosanoids in Biology and Pathophysiology (eds. Goetzl, E. J. & Lynch, K. R.) 188-208 (New York Academy of Sciences, New York, 2000)), cardiac function, (Himmel, H. M. et al. Evidence for Edg-3 Receptor-Mediated Activation of I(KACh) by Sphingosine-1-phosphate in Human Atrial Cardiomyocytes. Mol. Pharmacol. 58, 449-454 (2000)), blood leukocytes (Goetzl, E. J., Kong, Y. & Voice, J. K. Cutting Edge: Differential Constitutive Expression of Functional Receptors for Lysophosphatidic Acid by Human Blood Lymphocytes. J. Immunol. 164, 4996-4999 (2000)), and many more. The Edg family of GPCRs consists of 8 members, 5 of which (Edg-1, Edg-3, Edg-5, Edg-6 and Edg-8) respond to the phospholipid growth factor sphingosine-1-phosphate (SPP), whereas the remaining 3 (Edg-2, Edg-4 and Edg-7) respond to lysophosphatidic acid (LPA).

### SUMMARY OF THE INVENTION

The invention includes computational models and methods for structurally characterizing G-protein-coupled receptors (GPCRs), for ligand blocking (neutralizing) and receptor activating antibodies against GPCRs, and for producing such antibodies. Some of the preferred methods of the invention include computational models of GPCRs that facilitate the design of conformationally restricted peptides that are adapted to adopt the configuration of solvent-exposed (extracellular) loops to provoke a specific (antibody) immune response to the ligand binding domain of Edg-6. The ligand receptor domains of GPCRs are located on the extracellular (solvent exposed) region of the protein molecule. The employment of computational modeling in the inventions, to design peptides that adopt in solution the conformation of the extracellular protein loops, produce two types of extremely useful, inventive antibodies that have previously proven very difficult to develop, namely, those that bind to the receptor in such a way that they sterically block ligand binding to the active site and those that bind to the active site itself and activate the receptor.

It is the invention's ability to use a conformationally constrained peptide immunogen capable of binding ligand, as determined by nuclear magnetic resonance (NMR) and CD loop analysis, which is the significant step in the generation of a conformation specific antibody reagent tool that has functionality beyond simply binding a linear peptide sequence. The methods of the invention are a platform technology for producing ligand blocking or function blocking antibodies to GPCRs, among others, such as specifically targeting the LPA2 receptor for therapeutic use with ovarian and breast cancer.

Structural determination of GPCRs is a novel solution to the current lack of suitable tools to study GPCR biology. Experimental determination of accurate three-dimensional structures of membrane proteins is particularly difficult. Ten years of active investigation into the structure of rhodopsin (a GPCR) and related proteins (Schertler, G. F. X., Villa, C. & Henderson, R. Projection Structure of Rhodopsin. Nature 362, 770-772 (1993); Rees, D. C., Komiya, H., Yeates, T. O., Allen, J. P. & Feher, G. The Bacterial Photosynthetic Reaction Center as a Model for Membrane Proteins. Annu. Rev. Biochem. 58, 607-633 (1989)) have only recently resulted in its crystal structure at 2.8 Å resolution. (Palczewski, K. et al. Crystal Structure of Rhodopsin: A G Protein-Coupled Receptor. Science 289, 739-745 (2000)). 2D NMR studies on peptides with sequences that correspond to solvent-exposed regions of GPCR have also been used to characterize the structures of particular GPCR domains. (Mierke, D. F., Royo, M., Pellegrini, M., Sun, H. & Chorev, M. Peptide Mimetic of the Third Cytoplasmic Loop of the PTH PTHrP Receptor. J. Am. Chem. Soc. 118, 8998-9004 (1996); Yeagle, P. L., Alderfer, J. L. & Albert, A. D. Three Dimensional Structure of the Cytoplasmic Face of the G Protein Receptor Rhodopsin. Biochemistry 36, 9649-9654 (1997); Yeagle, P. L., Alderfer, J. L. & Albert, A. D. Structure of the Carboxyl Terminal Domain of Bovine Rhodopsin. Nature Struct. Biol. 2, 832-834 (1995); Yeagle, P. L., Alderfer, J. L. & Albert, A. D. Structure of the Third Cytoplasmic Loop of Bovine Rhodopsin. Biochemistry 34, 14621-14625 (1995); Yeagle, P. L., Alderfer, J. L. & Albert, A. D. Structure Determination of the Fourth Cytoplasmic Loop and Carboxyl Terminal Domain of Bovine Rhodopsin. Mol. Vis. 2 (1996); Yeagle, P. L., Alderfer, J. L. & Albert, A. D. The First and Second Cytoplasmic Loops of the G-Protein Receptor, Rhodopsin, Independently Form β-turns. Biochemistry 36, 3864-3869 (1997)). Such studies have demonstrated that peptide structures can be influenced by their environment, and in fact they often unfold in aqueous solution. Thus the incorporation of covalent cross-links are an important mechanism to bias the peptide structure toward the geometry it adopts when part of a larger protein structure. Other NMR studies applied to the characterization of solvent-exposed loops found in GPCR have demonstrated that conformational constraints imposed by cyclization and characterization in the presence of micelles both improve the structural stability of the peptides. (Mierke, D. F., Royo, M., Pellegrini, M., Sun, H. & Chorev, M. Peptide Mimetic of the Third Cytoplasmic Loop of the PTH PTHrP Receptor. J. Am. Chem. Soc. 118, 8998-9004 (1996)).

Modeling efforts have, until now, only attempted to elucidate membrane protein structures. Such extensive modeling efforts initially were based on the expectation that the transmembrane helices have a hydrophobic face oriented toward the membrane, and a more highly conserved face oriented inward to provide signal transduction capability. Modeling efforts based on this expectation have successfully been applied to predict an amino acid mutation that selectively influences agonist and antagonist binding to the cannabinoid GPCR receptor. (Bramblett, R. D., Panu, A. M., Ballesteros, J. A. & Reggio, P. H. Construction of A 3D Model of the Cannabinoid CB1 Receptor: Determination of Helix Ends and Helix Orientation. Life Sci. 56, 1971-1982 (1995); Tao, G. et al. Role of a Conserved Lysine Residue in the Peripheral Cannabinoid Receptor (CB₂): Evidence for Subtype Selectivity. Mol. Pharmacol. 55, 605-613 (1999)). More current modeling efforts use the rhodopsin GPCR crystal structure as a template, a procedure that provides reliable results for the transmembrane domains, which have high sequence homology as well as functional homology, thus supporting an assumption of three-dimensional homology. Sequence homology for the extracellular and intracellular loops, however, is much lower. Thus models developed for these regions are not generally reliable and therefore not applicable to other targets. This problem indicates the need for methods to structurally characterize the solvent-exposed and highly variable loop regions of GPCRs to design structurally defined antigens for the generation of polyclonal and monoclonal ligand blocking antibodies. Such methods are particularly relevant to GPCRs whose ligands interact with the extracellular loops as well as for the interaction of GPCRs with their intracellular G protein partners but can be expected to be applicable to other receptor systems which possess extracellular loops as part of the ligand binding domains is also relevant to protein receptors whose ligand binding domain is composed of dimer, trimers or multimers of the same or multiple different (heteromers) protein partners. This increased understanding of and ability to design peptides with biologically relevant conformations of these loops facilitates the development and commercialization of specific antibodies with therapeutic potential against GPCR.

An example of the preferred methods of the invention applies molecular modeling techniques to design appropriate cross-linking sites in peptides representing solvent-exposed segments of the S1P4 (previously called Edg6) receptor to allow the development of ligand blocking or stimulating monoclonal antibodies. This particular GPCR is important for three reasons. First, its specific expression in lymphoid cells and tissues (Gräler, M. H., Bernhardt, G. & Lipp, M. EDG6, a Novel G-Protein-Coupled Receptor Related to Receptors for Bioactive Lysophospholipids, Is Specifically Expressed in Lymphoid Tissue. Genomics 53, 164-169 (1998)) is indicative of a possible role in inflammatory responses and the immune system. Second, it currently has no known antagonist. Third, there are not currently any commercially available antibody research tools for this molecule.

The development of specific antibodies against many GPCRs has been seriously hindered by the lack of suitable immunogens. GPCRs are difficult to purify in significant amounts because they are membrane bound. It is difficult to synthesize active protein by conventional methods because of the hydrophobic nature of the membrane spanning regions in these proteins. The development of antibodies to peptide immunogens has been an alternative to using recombinant whole proteins as immunogens. The use of conventionally identified and produced peptide immunogens has yielded antibodies of restricted utility and their use in multiple assay formats has proven impractical or impossible. (Detheux, M. Orphan receptors: the search for new drug targets. Innovations in Pharm. Tech., 27-34 (2001)). The development of monoclonal antibodies to conventional peptide immunogens is not a practical solution, as these antibodies generally will only recognize an immunogen once it has been denatured, as in western blotting and will not recognize the 3D nature of the native protein. To date anti-GPCR peptide antibodies have demonstrated limited utility. The generation of polyclonal antibodies to peptides, while producing antibodies that often have a somewhat broader range of uses than monoclonal antibodies similarly produced, are limited due to batch size and variability among lots. Also, polyclonal antibodies have very limited, if any, therapeutic potential. The most useful class of anti-GPCR antibody would be a blocking or activating monoclonal antibody.

Both pharmaceutical and biotechnology companies are motivated to access cutting-edge technologies, which have the potential to vastly increase the speed and efficiency of the drug discovery process, now estimated at over $800 million to get a new drug to market. The obvious therapeutic nature of GPCR targets is something that all pharmaceutical and biotechnology companies recognize. The therapeutic nature of the S1P4 molecule as a target in drug discovery is two fold, first, GPCRs are a known family of receptor targets and second, the numerous Edg family members have been implicated in numerous pathological conditions. In addition, the presence of the S1P4 receptor primarily on immune system cells also offers a unique opportunity to target this growth factor receptor.

The market opportunity for novel methods of target prediction for GPCRs is also great. The methods of target generation for GPCRs result in tools for the generation of screening assays suitable for addressing S1P4. These methods are similarly applicable to other GPCRs as well.

Specific antibodies against the extracellular domains of Edg family of GPCRs have three major commercial applications; diagnostic assays, research tools for both basic research and drug discovery and their development into useful therapeutics. From the demonstration of receptor expression on the surface of recombinant cell types constructed for use on drug discovery platforms to providing evidence for the involvement of receptors in normal or abnormal physiology, and finally as therapeutic agents themselves (or in humanized form), these antibodies are invaluable tools with significant commercial potential. Antibodies have a wide range of applications for receptor studies such as flow cytometry, in situ binding studies/immunohistochemistry, western blot, immunoprecipitation and the development of diagnostic and therapeutic applications. The ability to generate panels of antibodies to GPCRs facilitates the development of assays useful in elucidating the function of these receptors. Ligand-blocking antibody(s) are particularly valuable for high throughput screening (HTS) of drug libraries produced by combinatorial chemistry, rational drug design or phage display.

It is therefore a primary object of this invention to provide computational models and methods for structurally characterizing G-protein-coupled receptors (GPCRs).

It is a further object of the invention to provide models and methods for producing ligand blocking (neutralizing) and receptor activating antibodies against GPCRs.

It is a further object of the invention to provide methods producing GPCR antibodies.

A preferred method of the invention for modeling G protein coupled receptors (GPCRs) and producing conformationally constrained peptides or fragments thereof, generally comprises the steps of: providing a molecular model of a GPCR and identifying a peptide sequence therein, having at least one amino acid residue involved in ligand binding; identifying amino acid sequences extracellular and proximal to at least one transmembrane domain and at least one extracellular loop; identifying at least one amino acid on said loop as an optimal location for a conformational constraint in said extracellular loop; mutating said identified amino acid to cysteine if not already cysteine; covalently connecting one or more linkers to said cysteine to conformationally constrain said peptide; and characterizing said constrained peptide using nuclear magnetic resonance (NMR) to verify a stable tertiary structure having conformations substantially similar to overlapping regions of a molecular model of said modeled GPCR containing said peptide.

The constrained peptide preferably has at least one interresidue carbon distance at at least one cross-link site, wherein the method further comprises the steps of, subjecting said constrained peptide to geometry optimization; subjecting said constrained peptide to molecular dynamic simulation to calculate a simulation-averaged distance between carbons at said cross-link site.

The step of the method of identifying preferably comprises the steps of, extracting at least one structure containing two transmembrane helical domains and their connecting extracellular loop; examining said loop to locate any potential cross-linking sites; and identifying at least one amino acid bearing side-chains directed between said two transmembrane helical domains that are close enough to form a disulfide cross-link if replaced by cysteine if cysteine is not already present suitable for same purpose of crosslinking.

The method may further comprise the steps of, deleting substantially all unidentified amino acids in said transmembrane domains, except for at least one amino acids proximate said extracellular loop; and adding one or more neutral caps.

The peptide of the method is preferably a G protein-coupled receptor (GPCR), wherein said GPCR is preferably an Edg receptor. Wherein said constrained peptide preferably has at least one interresidue carbon distance at at least one cross-link site, the method preferably further comprises the steps of, subjecting said constrained peptide to geometry optimization; subjecting said constrained peptide to molecular dynamic simulation to calculate a simulation-averaged distance between said carbons at said cross-link site; and comparing said simulation-averaged distances of said constrained peptide to corresponding distances of an unrestrained reference peptide to determine that said simulation-averaged distance of said constrained peptide are shorter than said corresponding distances of said reference peptide; wherein said constrained peptides comprises distances that are maintained with 10% of an original restraint distance prior to said molecular dynamics simulation.

The step of characterizing preferably results in heteronuclear 2D-nuclear overhauser effect (NOE) to allow derivation of said distance and comprises the step of determining any interaction between said constrained peptide and a molecule bearing at least one recognition element for a ligand, such as O-phosphoethanoloamine for S1P4, or a ligand analogue, and results in an NMR spectra comprising the recognition element, such as O-phosphoethanolamine, and either a Loop 1 B or Loop 3.

A preferred method for producing a GPCR antibody, generally comprises the steps of, providing a conformationally constrained GPCR peptide; coupling a carrier protein to said peptide to generate an antigen; immunizing an animal with said antigen to initiate production of an antibody to said antigen, wherein said carrier protein is preferably selected from a group consisting of keyhole limpet hemocyanin (KLH) and ovalbumin (OVA).

The method of producing an antibody may further comprise the step of purifying said antigen. The step of immunizing optimally comprises administering one or more adjuvants with said antigen. The antibody of the invention may be monoclonal or polyclonal.

### BRIEF DESCRIPTION OF THE DRAWINGS

Other objects, features and advantages will occur to those skilled in the art from the following description of the preferred embodiments and the accompanying drawings in which:

FIG. 1 is an image of sequences relative to LPA from the LPA₂ complex

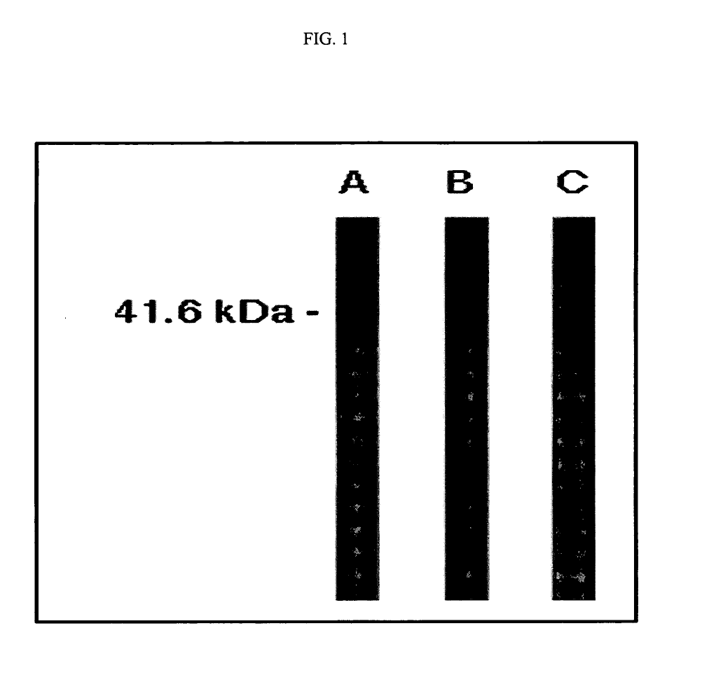

FIG. 2 is a Western Blot of monoclonal antibodies generated against the cyclic S1P4/Edg 6 cyclic molecular mimic, binding both endogenous (native S1P4) and CHO cells transfected with S1P4; and

FIG. 3 depicts the structures of SPP and O-phosphoethanolamine.

### DETAILED DESCRIPTION OF THE PREFERRED EMBODIMENTS AND METHODS

An example of a preferred method of the invention comprises an initial step of developing a model of the S1P4 receptor based on the validated model (Parrill, A. L. et al. Identification of S1P1/Edg1 Receptor Residues that Recognize Sphingosine 1-Phosphate. J. Biol. Chem. 275, 39379-39384 (2000); Parrill, A. L. et al. in Lysophospholipids and Eicosanoids in Biology and Pathophysiology (eds. Goetzl, E. J. & Lynch, K. R.) 330-339 (New York Academy of Sciences, New York, 2000); Bautista, D. L. et al. Dynamic Modeling of EDGI Receptor Structural Changes Induced by Site-Directed Mutations. J. Mol. Struct. THEOCHEM 529, 219-224 (2000)) of the Edg-1 receptor. The S1P4 receptor is a preferred receptor because there previously were no commercially available antibodies and it also has therapeutic potential against inflammatory and immune disorders due to its specific expression in lymphatic tissue. (Graler, M. H., Bernhardt, G. & Lipp, M. EDG6, a Novel G-Protein-Coupled Receptor Related to Receptors for Bioactive Lysophospholipids, Is Specifically Expressed in Lymphoid Tissue. Genomics 53, 164-169 (1998)). The S1P1 and S1P4 models share a key set of residues that have been demonstrated for the S1P1 receptor to be required for recognition and binding of sphingosine-1-phosphate (SPP). This set of residues includes two cationic amino acids (R120 and R292 in S1P1 which correlate to 121 and 287 in S1P4) as well as one anionic residue (E121 in S1P1, corresponding to 122 in S1P4). The model shows that the cationic residues interact with the phosphate group of SPP and that the anionic residue interacts with the cationic ammonium group of SPP. These modeled indicators are supported by experimental mutagenesis followed by radioligand binding and receptor activation assays that demonstrate the inability of receptors with alanine mutations at these key positions to bind or be activated by SPP. (Parrill, A. L. et al. Identification of Edg1 Receptor Residues that Recognize Sphingosine 1-Phosphate. J. Biol. Chem. 275, 39379-39384 (2000)).

A specific mutation of the S1P1E121 residue to Q will produce a receptor with selectivity for the glycerol-based phospholipid, LPA, instead of the sphingosine-based endogenous ligand, SPP. Graph 1 shows the ligand-induced GTP-γ-³⁵S binding assays of the wild-type S1P1 and S1P1E121Q mutant with both LPA and SPP. As this figure demonstrates, the model-based indicator of a change in receptor selectivity is indeed confirmed.

[image]

The homology modeling techniques used in this step assume that proteins sharing both significant sequence homology and functional homology also have homologous tertiary structure.

These Edg receptors do share common function, the transduction of signals from a common signaling molecule, sphingosine-1-phosphate. The Edg family of receptors also share significant sequence homology in the transmembrane domains (25-52%), with greater homologies between receptors specific for the same endogenous ligand. Their sequence homology in the loop regions that extend outside the membrane is significantly lower, however (15-39%), with no notable correlation between homology and ligand selectivity. Thus the design of conformationally constrained analogs to the extracellular and intracellular loop regions includes structural characterization of the loop and flanking residues from the reliable transmembrane domain of the model to predict cross-linking sites that provide peptide antigens with stable three-dimensional structure for antibody generation.

Molecular modeling is then used to determine the optimal location for a conformational constraint in the extracellular loops of S1P4. This modeling preferably begins by extracting structures containing two transmembrane helical domains and their connecting extracellular loop from our S1P4 model. These substructures are then visually examined to identify amino acids bearing side-chains directed between the two helical domains that were suitably close to form a disulfide cross-link if replaced by cysteine. This visual inspection identifies two possible cross-link sites in the first extracellular loop, two in the second, and only one in the third. Mutation of the amino acids to cysteine at the selected positions followed by covalent connection of the sulfur atoms to provide starting points for preliminary models of these five cross-linked peptides. Deletion of most amino acids in the transmembrane domains, with the exception of the two helical turns closest to the extracellular loop, and the addition of neutral caps (acetyl or NH₂) provides the preliminary models of these conformationally constrained peptides.

Next, modeling is used to evaluate the ability of the selected cross-links to maintain the modeled distance between the protein backbone atoms of the cross-linked amino acids. Each of the constrained peptides is subjected to geometry optimization to a root mean square gradient (RMSG) of 0.001 and molecular dynamics simulation. Dynamics simulations were run with 1 femtosecond time steps, equilibrated for 60 picoseconds, followed by data collection every 1

TABLE 1
Results of 100 ps molecular dynamics simulations on
constrained and unconstrained peptide sequences with two
dielectric constants (ε). Model references are average
distances for corresponding alpha carbons from a molecular
dynamics simulation on an unconstrained peptide.

| | Interresidue Alpha Carbon Distances (Å) at the | | | | |
|---|---|---|---|---|---|
| | Cross-link Site | | | | |
| | | | | Unconstrained | |
| | Model Reference | Constrained Loop | | Reference | |
| | ε = 1 | ε = 1 | ε = 80 | ε = 1 | ε = 80 |
| Loop 1 A | 9.0 | 6.6 | 4.8 | 9.6 | 31.6 |
| Loop 1 B* | 7.2 | 5.9 | 5.4 | 6.2 | 40.4 |
| Loop 2 A* | 6.5 | 5.8 | 5.7 | 7.2 | 26.8 |
| Loop 2 B | 7.0 | 6.4 | 5.4 | 6.1 | 27.5 |
| Loop 3* | 8.0 | 6.2 | 5.6 | 8.0 | 36.3 |

*Sequences shown in Table 2

picosecond for 100 picoseconds. The MMFF94 force field is preferably used due to its broad applicability to the functional groups found both in proteins and ligands. (Halgren, T. A. Merck Molecular Force Field. I. Basis, Form, Scope, Parameterization, and Performance of MMFF94*. J. Comp. Chem. 17, 490-519 (1996)). Each peptide is simulated with three separate solvent treatments. The first treatment of solvation is the default bulk dielectric of 1 (essentially no solvation). The second solvation treatment applies a bulk dielectric of 80 (aqueous solvation). The third solvation treatment includes a rectangular box of water molecules and applies periodic boundary conditions to promote bulk behavior. Reference peptides of the same length with the wild-type sequence are subjected to the same calculations. The calculations utilizing bulk dielectric constants rather than explicit water molecules are completed. Table 1 shows the simulation-averaged distances between the alpha carbons of the amino acids at the cross-link site.

The results shown in Table 1 demonstrate that the unconstrained reference peptides have a significant tendency to unfold in polar environments (alpha carbon distances >20 Å). This is an expected result due to replacement of intramolecular electrostatic interactions with equivalent and statistically favored intermolecular interactions between the peptide and water. These steps also indicate that the cross-link site modeled in the Loop 1 B peptide maintains a distance closer to its reference than does the Loop 1 A peptide. Similarly, the Loop 2 A peptide is more similar to its reference than is the Loop 2 B peptide. There is also a significantly smaller change due to changes in solvation treatment among the constrained peptides. Thus the peptides marked with an asterisk are those preferably selected for subsequent characterization by NMR and for use in the development of antibodies.

Circular dichroism spectra of the Loop 1 B peptide (peptide from New England Peptide) in aqueous solution with and without sodium dodecyl sulfate (SDS) micelles are shown in Graph 2. These spectra show that the peptide is more structured in the presence of micelles as evidenced by the absorptions at 205 and 220 nm. Thus additional characterization of this peptide by NMR

utilizes perdueterated SDS micelles.

The invention utilizes a non-blocking non-activating monoclonal antibody to the human S1P4/Edg-6 protein (from Exalpha Biologicals in Watertown, Mass.) that was developed using standard, non-constrained/non-loop peptide immunogen, whereby the antibody recognizes a linear amino acid sequence near the C terminus of the native S1P4/Edg-6 molecule. The antibody is employed in the methods of the invention as a positive antibody control in the generation of the monoclonal antibodies because it functions well in ELISA (on fixed S1P4/Edg-6 transfected cells) and western blot.

[image]

Peptide Design and NMR Characterization

As noted, after the molecular model of the S1P4/Edg 6 receptor is developed, the next step is to incorporate covalent cross-links into the peptide structures to bias their three-dimensional structures toward the geometry adopted by the peptide as part of the intact receptor. This step began using computational modeling of peptides containing covalent cross-links at different positions. The best model is characterized by NMR to demonstrate its suitability as a conformationally stable antigen. The modeling step determines whether cross-link sites maintain the structure of residues analogous to the transmembrane domain (termini of the peptide analog). However, the modeling alone does not sufficiently characterize the structure of the loop connecting those residues. NMR is used to characterize the entire peptide structure, allowing comparison of peptide termini to the original model.

As noted, proteins within the Edg family share significant sequence homology in the transmembrane domains but vary in the extracellular regions. These differences are exploited to develop specific antibodies against each protein in the family, given a good source of loop-mimicking peptides to use as immunogen. The computational modeling uses the preceding molecular modeling to engineer loop-mimicking peptides. The novelty of this approach is that specific distances from the model are used to select an optimal cross-link site with a very limited range of conformations. This approach significantly improves on prior cross-linking strategies that utilizes linkers containing multiple flexible bonds, which NMR has demonstrated did not induce a stable three-dimensional structure. (Mierke, D. F., Royo, M., Pellegrini, M., Sun, H. & Chorev, M. Peptide Mimetic of the Third Cytoplasmic Loop of the PTH PTHrP Receptor. J. Am. Chem. Soc. 118, 8998-9004 (1996)). The peptides are designed to include sites that can be covalently connected to generate conformationally restricted peptides representing the extracellular loops of the S1P4/Edg-6 receptor. The covalent connection site is designed to promote adoption of the same 3D structure in solution as the loop adopts in the intact receptor. This allows for the development of monoclonal antibodies that will recognize the receptor in cell membranes. For S1P4, NMR is used to characterize the loop peptides and their interaction with a molecule bearing the recognition elements from the polar headgroup of sphingosine-1-phospate (SPP, the endogenous ligand for S1P4/Edg-6), namely O-phosphoethanolamine. Similar approaches can be employed for other receptors using either the natural ligand or ligand analogues. It can also be anticipated that agonists or antagonists can be used to generate models of activated or inactive receptors to drive the generation of specific blocking or activating antibodies. NMR then provides distance and dihedral constraints that computational modeling utilizes to generate accurate 3D models of the extracellular loops. Thus the method uses computational modeling to drive the design of peptides that adopt the conformation of interest. This model-driven peptide design represents a novel method developed here and will be widely applicable for developing antibodies against GPCRs and other membrane proteins.

Structurally Characterizing the Conformation of the Extracellular Loops of S1P4

Of the 5 cross-linked peptides evaluated that correlate to the three extracellular loops of S1P4 (formerly know as Edg6), three of these peptides are used in the example to characterize the 3D molecular structure to generate antibody reagents to these regions. Molecular modeling is applied to determine the optimal location for a conformational constraint in peptides designed to

TABLE 2

| Extracellular | |
|---|---|
| Loop 1 | |
| Wild type sequence | TGAAYLANVLLSGARTFRLAPAQWFLREGLLFT |
| Loop 1 B | TGAYLANVLLSGARTFRLAPAQWFLREGCLFT |
| Extracellular | |
| Loop 2 | |
| Wild type sequence | AALLGMLPLLGWNCLCAFDRCSSLLPLYSKRYILFCLV |
| Model peptide 2 A | AALGMLPLLGWNALAAFDRASSLLPLYSKRYILFLV |
| Extracellular | |
| Loop 3 | |
| Wild type sequence | FLVCWGPLFGLLLADVFGSNLWAQEYLRGMDWILALAVL |
| Model peptide 3 | FLVWGPLFGLLLADVFGSNLWAQEYLRGMDWILAAVL |
| Peptide sequences representing the Edg-6 extra-cellular loops used in NMR. Regions underlined with a single line in sequences show extracellular ends of the helices connected by the loop as predicted by the modeling analysis. Amino acids underlined with a double line are those residues that correspond to the validated Edg-1 residues predicted to interact with the charged ammonium and phosphate moieties of SPP. Boxed residues represent cross-link sites that best maintain the distance between | |
| # those residues in the model. | |

mimic the conformation of the solvent-exposed extracellular loops of S1P4.

The results of the molecular modeling to determine optimal location for a conformational constraint in the extracellular loops of S1P4 are described further below.

The designed peptides are structurally characterized by NMR to verify a stable tertiary structure with conformations consistent with the molecular model for overlapping regions. First, the production of peptides is directed to mimic the S1P4 extracellular loops, as shown in Table 2, from commercially available custom synthesis sources at 95% purity or greater. Circular dichroism (CD) is initiated to determine the conditions that produce at least the expected alpha-helical content based on the inclusion of residues at each termini from the alpha-helical transmembrane domains. CD spectra are collected in aqueous phosphate buffer with and without detergents above their critical micellar concentration. Sodium dodecyl sulfate (SDS) and dodecyl phosphocholine are two detergents that are applied to the characterization of GPCR loop peptides. As shown by the data, Loop 1 B is unstructured in aqueous buffer, but shows absorption at 220 nm characteristic of alpha-helical structure. Next, NMR is performed with suppression of the water peak on a 500 MHz spectrometer. The general procedure for characterization of each peptide first involves the collection of one-dimensional ¹H spectra on 1 and 2 mM samples buffered at pH values from 5 to 7 at temperatures ranging from 25-45° C. When CD results indicate that micelles are present, then the samples also should contain between 200 and 300 mM perdeuterated detergent (sodium dodecylsulfate or dodecylphosphocholine). The results are used to determine the conditions that yield optimal signal resolution and concentration independence. A lack of concentration independence indicates peptide aggregation and requires characterization of more dilute solutions. Second, two-dimensional correlation spectroscopy (COSY), totally correlated spectroscopy (TOCSY) (with 40 and 70 ms mixing times) and nuclear overhauser effect (NOE) (with 80 and 160 ms mixing times) spectra are obtained for samples at the previously determined optimal pH and temperature in order to fully assign the resonances. When complete assignment of the resonances is confounded by signal overlap, data is collected at multiple temperatures and pH values. Next, NOE spectra with mixing times of 80, 160, 240 and 320 ms are collected to generate distance restraints for model development. The rate of NOE buildup as a function of mixing time in the linear region is used to derive distances rather than the NOE volumes at a single mixing time to insure that effects from spin diffusion are not included. (Wuthrich, K. NMR of Proteins and Nucleic Acids (John Wiley & Sons, New York, 1986)). The relationship between NOE buildup rate and distance is calibrated using several known distances (for example, the distance between vicinal protons on the aromatic ring of phenylalanine). The distances obtained in this fashion are used as distance restraints during simulated annealing simulations. A family of structures are obtained from this procedure. Each structure in this family is then geometry optimized without distance restraints. Structures that maintain distances within 10% of the original restraint are averaged together to produce a single model that will again be geometry optimized and checked for consistency with the original distance restraints. (Wuthrich, K. NMR of Proteins and Nucleic Acids (John Wiley & Sons, New York, 1986)).

The determined structures of the loop-mimicking peptides are compared with the experimentally validated theoretical model of the transmembrane domain to determine if the characterized peptide analog structure is compatible with that of the entire protein. When the termini of the peptide superpose well on the analogous residues from the transmembrane domain, a more complete model for the S1P4 receptor results.

NMR is used to characterize the interactions between the extracellular loops and O-phosphoethanolamine, a mimic of the S1P4 endogenous ligand to determine if potential for ligand binding exists.

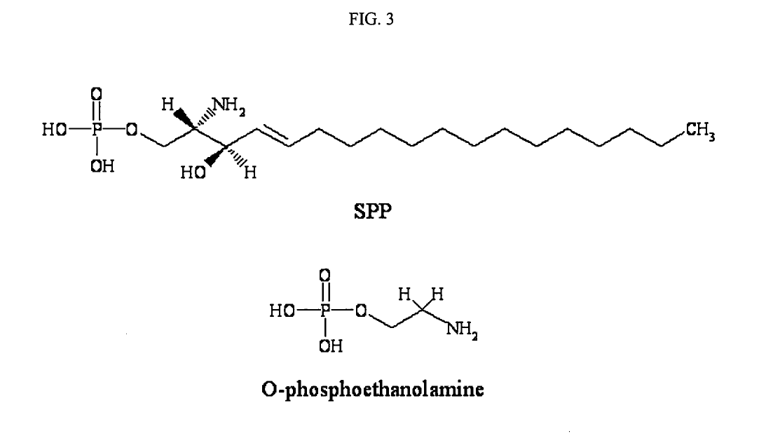

Key interactions between SPP and its ligands are identified by the modeling studies on the S1P1 receptor and validated by binding and functional studies of site directed mutants of the S1P1 receptor. Graph 1 shows the interactions between S1P1 and SPP, and FIG. 3 depicts the structures of SPP and O-phosphoethanolamine. The most significant interactions are between charged amino acids in the receptor (R120, E121 and R292 in Edg-1 corresponding to R121, E122 and R287 of S1P4) at the top of transmembrane helices three and seven and the charged groups (phosphate and ammonium) of SPP. The first extracellular loop peptide (Loop 1 B) to be characterized and used in antibody development includes two of these charged residues (R121 and E122). The third extracellular loop peptide (Loop 3) contains the remaining charged residue. Thus the loop peptides should bind a molecule that contains the charged functional groups of SPP, but lacking the hydrophobic tail. Such binding indicates that the monoclonal antibodies against these peptides could act as ligand blocking antibodies.

NMR spectra of mixtures containing O-phosphoethanolamine and either Loop 1 B or Loop 3 reflect ligand binding. The most important NMR result for the characterization of the interaction between the constrained peptides and O-phosphoethanolamine are heteronuclear 2D-NOE that allow derivation of the distances between the cationic nitrogens of R121 and R287 and the phosphate phosphorous atom of O-phosphoethanolamine. Additional information is derived from chemical shift changes of the protons in residues of the loop peptides upon addition of the phosphoethanolamine as well as chemical shift changes of protons on the phosphoethanolamine. Such chemical shift changes are indicative of changes in the electronic environment of these protons and are used in high-throughput NMR assays of ligand binding. (Shuker, S. B., Hajduk, P. J., Meadows, R. P. & Fesik, S. W. Discovering High-Affinity Ligands for Proteins: SAR by NMR. Science 274, 1531-1534 (1996)).

Table 3 below shows the modeled sequence of human S1P4 Extracellular Loop 1 using the preferred method of the invention. This modeled sequence of human S1P4 is used to synthesize the molecular mimic cyclic S1P4 construct. The cyclic mimic was constrained by the incorporation of a disulfide bond between the two introduced cysteine residues (shown as boxed ° C.'s in the table below) in the Loop 1B structure. This new cyclic peptide mimic demonstrated structure was indicated using CD loop analysis.

The synthesized cyclic mimic also has a C-terminal cysteine residue incorporated into it which is used to couple to KLH (keyhole limpet hemocyanin) through standard chemistries with which those skilled in the art will be familiar.

The KLH-cyclic S1P4 mimic is immunized in balb/c mice using standard immunization procedures. Mouse serum is collected and titer verses unconjugated peptide is preferably determined using standard direct ELISA methodology. The mice demonstrating the highest titers verses peptide mimic are sacrificed and the spleens harvested and spleenocytes isolated and fused to a suitable fusion partner using standard fusion techniques originally described by Kohler and Milstein in 1974.

TABLE 3

| | |
|---|---|
| Extracellular | |
| Loop 1 | |
| Wild type | TGAAYLANVLLSGARTFRLAPAQWFLREGLLFT |
| sequence | |
| Loop 1 B | TGAYLANVLLSGARTFRLAPAQWFLREGLFT |

As a further examples, below are two modeled cyclic peptides with potential for use as LPA 2 (Edg4) cyclic receptor mimics for the purpose of generating blocking or activating monclonals to LPA₂. LPA₂ receptor peptides potentially useful as extracellular antigens and the amino acids involved in interactions with LPA were modeled and are shown in bold below.

Loop 1
AYLFLMFHTGPRTARLSLEGWFLRQGLLD
Loop 2
LGLLPAHSWHCLCALDRCSRMAPLLSRSYLAVWAL
Loop 3
CWTPGQVVLLLDGLGCESCNVLAVEKYFLLLA

Loop 1 is suitable for intramolecular disulfide constraint such as:
AYLFLMFCTGPRTARLSLCGWFLRQGLLD

Loop 2 is suitable for intramolecular disulfide constraint such as
LGLLPAHCWHSLSALDRSSRMAPLLCRSYLAVWAL

Loop 3 is unsuitable for only a single disulfide bond to span the ends of flanking transmembrane (TM) domains as the ends are too far apart for a single disulfide to span. Other spacer chemistries could be employed to span this region. In such instances, other standard chemistries are applied to introduce spacers, such as carbon spacers interposed between disulfides, of varying lengths that would allow for the antigen binding 3D structure of this loop to be maintained and hence to be a suitable immunogen for monoclonal antibody development. Cross linking may be achieved using a variety of chemistries, including spacer atoms, that allows for flexibility as to where the linker/s is placed and as to the distances that can be achieved and/or maintained in the loop structure. There are also commercially available cross linkers (Pierce) that contain, for example, a bifunctional linker and 5 or 10 carbon spacers. This linker could be used instead of a direct cross linking of two cysteine residues on the peptide strand.

The ascending and descending portions of the extracellular loops need not be from the same loop (i.e. not directly contiguous but separated by another loop, transmembrane, or other domains). The active or binding site may consist of amino acids on separate loops in the native GPCR protein. Using the methods of the invention, the loops may be linked with a series of amino acids either composed of amino acids from one or the other or both of the newly linked loops. The active residues would then be maintained in the active 3D orientation.

Preferred Methods for Antibody Development to Edg GPCR peptides

Conjugation of conformationally constrained peptides to conventionally used carrier proteins such as keyhole limpet hemocyanin (KLH) and ovalbumin (OVA) is performed to generate the antigen used to immunize animals. This antigen is used for the generation of both polyclonal and monoclonal antibodies.

Conventionally used carrier proteins such as KLH and OVA are coupled to the conformationally restricted peptides. Coupling is conducted by traditional glutaraldehyde chemistry or other feasible biochemical procedures. The conjugates are then purified through size-exclusion gel filtration chromatography and/or dialysis and used in conjunction with adjuvants for immunizations.

Mice (for monoclonal antibody production) and rabbits (for polyclonal antibody production) are immunized with the KLH conjugated peptide antigens in adjuvant. Rabbits (New Zealand White, HsdOkd:NZW) and mice (balb/c) are preferably utilized.

Preferred Method for Polyclonal Antibody Production

New Zealand white rabbits are immunized with KLH conjugated peptide following established immunization protocols. All immunizations and bleeds are preferably carried out with immunogen available from Exalpha Biologicals, Inc., Watertown, Mass. Serum samples are collected and frozen for evaluation. Pre-bleeds are extracted from the rabbits prior to an initial primary immunization. The rabbits are subjected to a series of six booster immunizations following the primary immunization. Post-immunization bleeds are obtained in conjunction with the last three immunizations. The bleeds are screened for adequate antibody titers towards the ovalbumin conjugated peptide by ELISAs. A rabbit polyclonal antibody to a linear peptide sequence from the C terminus of human S1P4 is commercially available from Exalpha Biologicals, Inc., Watertown, Mass. This antibody is used as a positive control in screening experiments along with appropriate negative control rabbit sera.

Preferred Methods for Monoclonal Antibody Production

BALB/c mice are immunized with KLH conjugated peptide in adjuvant. The mice are sacrificed, and the cellular lymphocytes harvested. Hybridomas are generated following established protocols (Kohler, G. & Milstein, C. Continuous cultures of fused cells secreting antibody of predefined specificity. Nature 256, 495-497 (1975)) with the exception of performing the selection and cloning of the hybridomas in “Clonacell-HY” medium. (Davis, J. M., Pennington, J. E., Kubler, A. M. & Conscience, J. F. A simple, single-step technique for selecting and cloning hybridomas for the production of monoclonal antibodies. J. Immunol. Methods 50, 161-171 (1982); Civin, C. I. & Banquerigo, M. L. Rapid, efficient cloning of murine hybridoma cells in low gelation temperature agarose. J. Immunol. Methods 61, 1-8 (1983)). Positive clones are identified by HAT (hypoxanthine/aminopterin/thymidine) selection, and cultured in selective media. Positive hybridoma culture supernatants are screened for a specific antibody response in ELISAs. Putative positive hybridomas are sub-cloned on Clonacell-HY medium, and confirmed for monoclonality and stability. The monoclonal antibodies generated from robust hybridomas are then characterized, e.g., ELISA, flow cytometry and Western immunoblots and for function blocking or activating activities in the GTP-γ-³⁵S binding assay (a functional assay for ligand binding to GPCR and signal transduction through G proteins).

Characterization of Immune Sera

The following preferred screening steps are preferably used to characterize the S1P4 antibodies produced in response to the model-driven peptide design for any number of uses including use as research tools and potential therapeutic leads.

1. GTP-γ-³⁵S Binding Assay is Applied to Test for Function Blocking or Activating Antibody.

Anti-S1P4 antibodies are tested for their ability to block ligand activation of S1P4 as tested in the GTP-γ-³⁵S assay, a functional assay for ligand-induced activation of GPCRs. Similarly, all antibodies should be tested for their ability to activate the S1P4 receptor.

Membrane preparations of recombinant cell lines expressing the human S1P4 receptor (available from Exalpha Biologicals, Inc., Watertown, Mass.) suitable for GTP-γ-³⁵S binding assays (Barr, A. J., Brass, L. F. & Manning, D. R. Reconstitution of receptors and GTP-binding regulatory proteins (G proteins) in Sf9 cells. A direct evaluation of selectivity in receptor G protein coupling. J Biol Chem 272, 2223-2229 (1997); Windh, R. T. et al. Differential Coupling of the Sphingosine 1-Phosphate Receptors Edg-1, Edg-3, and H218/Edg-5 to the G(i), G(q), and G(12) Families of Heterotrimeric G Proteins. J. Biol. Chem. 274, 27351-27358 (1999); Sim, L. J., Selley, D. E. & Childers, S. R. Autoradiographic visualization in brain of receptor-G protein coupling using [³⁵S]GTP gamma S binding. Methods Mol. Biol. 83, 117-132 (1997)) are employed to screen antibodies generated for their ability to block the ligand (SPP)-receptor interaction. Control antibodies are assayed simultaneously to determine background levels in all assays. Additionally, the ability of antibodies to activate the S1P4 receptor and signal through relevant G protein are preferably assessed.

2. Western Blot on Cell Lysates of S1P4 Transfected Cells is Applied to Test for Applicability of Antibodies as Tools to Analyze S1P4 Receptor Expression.

Cell lines that express S1P4 are lysed and fractionated through SDS-polyacrylamide gel electrophoresis (lysate available from Exalpha Biologicals, Inc., Watertown, Mass.). The fractionated proteins are transferred onto nitrocellulose blotting membranes. The membranes are exposed to the generated monoclonal or polyclonal antibody preparations and positive and negative control antibodies, and then detected with a secondary goat anti-mouse or anti-rabbit HRP labeled conjugate. The blots are exposed to a fluorometric substrate (Pierce femto-signal or other suitable substrate), and positive reactivity identified by the development and presence of a specific banding profile on the blots indicating reactivity with a protein of S1P4 molecular weight.

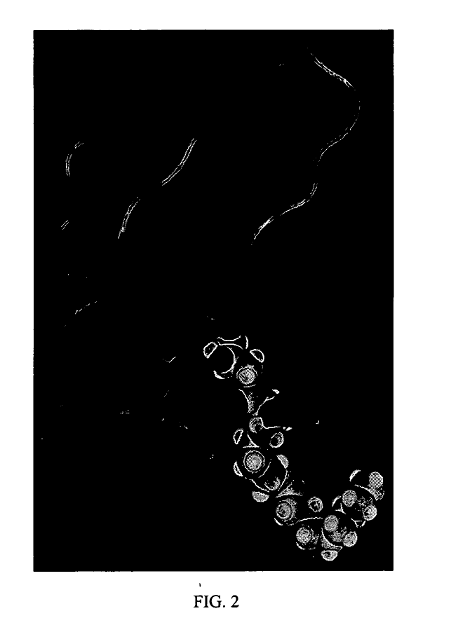

FIG. 2 illustrates a Western Blot analysis of monoclonal antibodies generated against the cyclic S1P4 cyclic molecular mimic binding both endogenous (native S1P4) and CHO cells transfected with S1P4. The Western blot was performed using 50 μg/lane of lysate prepared from S1P4 transfected CHO cells (or control mock transfectd CHO cells) with Laemmli sample buffer and heat 10 min at 90° C. Gels are transferred to nitrocellulose blocked with 2% non-fat dry milk in tris buffered saline and primary antibodies are added for 1 hour with shaking at room temperature. Incubation with the secondary antibody (HRP-conjugated goat anti-mouse heavy and light chain antibody is added for 1 h at room temperature with constant shaking. The blots are washed with tris tween×4 and developed with Pierce's West Femto™ chemluminescent detection system.

Clone 26 binding CHO cells transfected with full length functional human S1P4 [lane A], CHO mock transfected cells with clone 26 [lane B] and S1P4 transfected CHO cells blotted with Exalpha positive control anti-human S1P4 monoclonal antibody catalog (catalog no. X1533M) [lane C]. This clone, number 26, demonstrates no agonist or antagonist activity when screened in the phospho ERK 1/2 assay (another secondary readout for ligand induced S1P4 receptor activation). It may bind only a denatured epitope on SDS PAGE gels.

3 S1P4 Transfected Cell Lines are Screened by Flow Cytometry to Determine Cell-Surface Bound Reactivity.

The generation of antibodies reactive against GPCRs from the mice and rabbits immunized with the peptide conjugates is determined using standard procedures for flow cytometry (FCM). Cell lines that are stably or transiently transfected with S1P4 e.g. CH0 or RH7777 (available from Exalpha) are incubated with serial dilutions of the putative anti-S1P4 antibodies or positive and negative control antibodies. The antibodies are detected using a goat anti-mouse-FITC or goat anti-rabbit-FITC labeled second antibody and subjected to FCM analysis. The immunofluorescence histograms are examined for indications of positive antibody reactivity above control levels.

4. Antibodies are Screened for Positive Reactivity to the S1P4 Cyclic Mimic Antigen by Direct ELISA.

Monoclonal and polyclonal antibody preparations are incubated at serial dilutions in microtiter plates coated with the appropriate S1P4-OVA peptide or control OVA-peptide. Such peptide OVA and KLA conjugates can be synthesized by those skilled in the art based on the sequence disclosed herein. The antibodies are identified by a secondary goat anti-mouse or anti-rabbit—HRP labeled conjugate. The reactivity is determined through colorimetric processing, and the optical density measured. Monoclonal and polyclonal anti-S1P4 antibodies (available from Exalpha Biological, Inc., Watertown, Mass.) are used as positive controls. These antibodies have been demonstrated to be S1P4 specific in cell-based ELISA and western blot against panels of Edg transfected cell lines (human Edg-1, 2, 3, 4, 5, 6, 7, rat 8).

For example, CHO cells that are stably transfected with human S1P4 (formerly Edg 6) a GPCR (g protein coupled receptor) for sphingosine 1-phosphate (S1P) a lipid growth factor mediator or mock transfected CHO control cells are washed in DMEM and starved of serum for 4 hours at 37° C.

One million cells in 1 ml serum free DMEM were challenged with monoclonal antibodies that had been raised against a S1P4 cyclic molecular mimic loop 1B structure described herein for 10 minutes at room temperature. Monoclonal antibody supernatants containing DMEM with 10% fetal bovine serum antibiotics, L glutamine media supplements, were pretreated with cell culture tested Norite™ (Sigma Chemical Company) for 24 hours at 4 C to remove endogenous S1P from the media prior to the addition of supernatant to cells. Following antibody addition and incubation, 10 nM S1P was added for 15 minutes at 37 C. Controls of media alone, media and Norite™, media and 10 nM S1P, media-Norite™ and SIP, and media PMA were included.

[image]

The Graph 3 above shows the results as a percent of control (percent of 10 nM SIP challenged cells).

Similarly, Table 4 below shows the same results including CHO mock transfected control cells, presented as ng/ml phospho ERK 1/2 (as measured in commercially available R&D Systems, Inc. phospho ERK 1/2 ELISA).

TABLE 4

| | CHO EDG6 | CHO |
|---|---|---|
| | (S1P4) | Control |
| Medium (serum free DMEM) | 0.160 | 0.000 |
| Norite | 0.150 | 0.080 |
| 10 nM S1P | 1.280 | 0.000 |
| 10 nM S1P + Norite | 0.160 | 0.000 |
| Clone #1 | 0.410 | 0.030 |
| Clone #3 | 0.410 | 0.000 |
| Clone #9 | 0.480 | 0.070 |
| Clone #11 | 0.690 | 0.000 |
| Clone #23 | 4.070 | 0.030 |
| Clone #30 | 4.160 | 0.070 |
| Clone #31 | 4.590 | 0.000 |
| Clone #34 | 4.760 | 0.000 |

Modifications of the methods, models, antigens, and antibodies of the invention will occur to those skilled in the art and are within the following claims:

1. A method for modeling G protein coupled receptors (GPCR) and producing conformationally constrained peptides or fragments thereof, comprising the steps of:
providing a molecular model of a GPCR and identifying at least one peptide sequence therein having at least one peptide residue involved in ligand binding;
identifying at least one amino acid sequence extracellular and proximal to at least one transmembrane domain and at least one extracellular loop;
identifying at least one amino acid on said loop as an optimal location for a conformational constraint in said extracellular loop;
mutating said identified amino acid to cysteine if not already cysteine;
covalently connecting one or more linkers to said cysteine to conformationally constrain said peptide; and
characterizing said constrained peptide using nuclear magnetic resonance (NMR) to verify a stable tertiary structure having conformations substantially similar to overlapping regions of a molecular model of said modeled GPCR comprising said peptide.

2. The method of claim 1, wherein said constrained peptide has at least one interresidue carbon distance at at least one cross-link site, further comprising the steps of,
subjecting said constrained peptide to geometry optimization; and
subjecting said constrained peptide to molecular dynamic simulation to calculate a simulation-averaged distance between carbons at said cross-link site.

3. The system of claim 1, wherein said step of identifying comprising the steps of,
extracting at least one structure containing two transmembrane helical domains and their connecting extracellular loop;
examining said loop to locate any potential cross-linking sites;
identifying at least one amino acid bearing side-chains directed between said two transmembrane helical domains that are close enough to form a disulfide cross-link if replaced by cysteine.

4. The method of claim 1 further comprising the steps of,
deleting substantially all unidentified amino acids in said transmembrane domains, except for at least one amino acids proximate said extracellular loop; and
adding one or more neutral caps.

5. The method claim 1, wherein said peptide is a G protein-coupled receptor (GPCR).

6. The method of claim 5, wherein said GPCR is an Edg receptor.

7. The method of claim 6, wherein said Edg receptor is S1P4.

8. The method of claim 1, wherein said constrained peptide has at least one interresidue distance at at least one cross-link site, further comprising the steps of,
subjecting said constrained peptide to geometry optimization;
subjecting said constrained peptide to molecular dynamic simulation to calculate a simulation-averaged distance at said cross-link site; and
comparing said simulation-averaged distances of said constrained peptide to corresponding distances of an unrestrained reference peptide to determine that said simulation-averaged distance of said constrained peptide are shorter that said corresponding distances of said reference peptide.

9. The method of claim 8, wherein said constrained peptides comprises distances that are maintained with 10% of an original restraint distance prior to said molecular dynamics simulation.

10. The method of claim 1, wherein said constrained peptide comprises at least one interresidue distance at at least one cross-link site, and wherein said step of characterizing results in heteronuclear 2D-nuclear overhauser effect (NOE) to allow derivation of said distance.

11. The method of claim 1, wherein said step of characterizing comprises the step of determining any interaction between said constrained peptide and at least one molecule bearing at least one recognition element for a ligand, or a ligand analogue, and results in an NMR spectra comprising the recognition element.

12. The method of claim 1, wherein said characterizing step results in an NMR spectra comprising a ligand or ligand analogue.

13. The method of claim 1, wherein at least one of said linkers is a sulfur atom.

14. The method of claim 1, wherein a plurality of extracellular loops from a single GPCR are cross linked.

15. A method for producing a GPCR antibody, comprising the steps of,
providing a conformationally constrained GPCR peptide;
coupling a carrier protein to said peptide to generate an antigen;
immunizing an animal with said antigen to initiate production of an antibody to said antigen.

16. The method of claim 15, wherein said carrier protein is selected from a group consisting of keyhole limpet hemocyanin (KLH) and ovalbumin (OVA).

17. The method of claim 15, further comprising the step of purifying said antigen.

18. The method of claim 15, wherein said step of immunizing comprises administering one or more adjuvants with said antigen.

19. The method of claim 15, wherein said antibody is monoclonal or polyclonal.

20. The method of claim 15, wherein said GPCR peptide is an Edg receptor peptide.

21. The method of claim 20, wherein said Edg receptor is S1P4.

22. The method of claim 15, wherein said antigen is a cyclic SIP mimic.

23. The method of claim 13, wherein said antibody is an anti-S1P4 antibody.

24. An antibody produced using the method of claim 15.
